(19) United States
(12) Patent Application Publication
(10) Pub. No.: US 2026/0137919 A1
(43) Pub. Date: May 21, 2026

(54) Needle module for a hand-held device for locally puncturing a human or an animal skin, hand-held device and method for operating a needle module

(71) Applicant: MT.DERM GmbH, Berlin (DE)

(72) Inventor: Dirk Scherkowski, Berlin (DE)

(73) Assignee: MT.DERM GmbH, Berlin (DE)

(21) Appl. No.: 19/367,480

(22) Filed: Oct. 23, 2025

(30) Foreign Application Priority Data
Oct. 24, 2024 (EP) 24208709.6

(51) Int. Cl. A61M 37/00

(52) U.S. Cl. CPC A61M 37/0076; A61M 2205/103; A61M 2250/00

(57) ABSTRACT

A needle module for a hand-held device for locally puncturing a human or an animal skin. The needle module having a module housing including a module tip with a front-side module opening. A puncturing device having a puncturing needle which is arranged on a needle shaft and has a needle tip is received in the module housing. The puncturing needle can be linearly displaced between a retracted position and an extended position. A transmission device is at least partially received in the module housing and transmits a drive movement to the needle shaft. The transmission device has a rolling device having a first rolling component and a second rolling component. During the transmission of the drive movement, the first rolling component rolls on a rolling surface on the second rolling component. Furthermore, a hand-held device and a method for operating a needle module are provided.

The invention relates to a needle module for a hand-held device for locally puncturing a human skin or an animal skin, a hand-held device for locally puncturing a human skin or an animal skin and a method for operating a needle module.

### BACKGROUND

Such needle modules are used in hand-held devices for locally puncturing a human or an animal skin in order to provide a puncturing device with which the skin can be punctured locally repeatedly. The local puncturing of the skin can serve in particular to introduce a dye, a medical and/or a cosmetic active substance into the skin. However, such hand-held devices are also used without depositing substance into the skin in order to stimulate the skin locally.

In addition to the needle module, the hand-held device for locally puncturing the skin regularly has a drive device with which a drive movement or drive force is repeatedly provided for operating the puncturing device of the needle module, in particular in order to displace one or more puncturing needles of the puncturing device between a retracted position and an extended position. In this way, a needle tip of the puncturing needle can be repeatedly punctured locally into the skin and retracted out of the skin during operation.

### SUMMARY

The object of the invention is to specify a needle module for a hand-held device for locally puncturing a human skin or an animal skin, a hand-held device for locally puncturing a human skin or an animal skin and a method for operating a needle module, in which a provided drive movement can be transmitted to a puncturing device in an efficient and operationally reliable manner.

To achieve this, a needle module for a hand-held device for locally puncturing a human or an animal skin and a hand-held device for locally puncturing a human or an animal skin according to independent claims 1 and 15 are provided. Furthermore, a method for operating a needle module according to independent claim 16 is provided. Embodiments are the subject matter of dependent claims.

According to one aspect, a needle module for a hand-held device for locally puncturing a human skin or an animal skin is provided which comprises the following: a module housing which has a module tip and a front-side module opening formed in the region of the module tip; a puncturing device which has at least one puncturing needle which is arranged on a needle shaft and has a needle tip, which puncturing device is received in the module housing in such a way that the at least one puncturing needle can be displaced between a retracted position and an extended position by means of a linear movement, wherein the at least one puncturing needle extends through the front-side module opening at least in the extended position and the needle tip is arranged outside the module housing in this case; and a transmission device. The transmission device is at least partially received in the module housing and can be operated to transmit a drive movement, which is provided on a drive side with a repetition frequency, to the needle shaft in such a way that the transmitted drive movement can be coupled on an output side to the at least one puncturing needle in order to displace the latter repeatedly between the retracted position and the extended position. The transmission device has a rolling device which has a first rolling component, which is mounted rotatably, and a second rolling component and onto which rolling device the drive movement which is provided on the drive side can be introduced in such a way that, during the transmission of the drive movement, a rolling movement is carried out in which the first rolling component rolls on a rolling surface on the second rolling component.

According to a further aspect, a hand-held device for locally puncturing a human skin or an animal skin is provided which comprises the abovementioned needle module and a drive device which is functionally coupled to the needle module in order to transmit a drive movement, which is provided by the drive device, to the needle module.

According to a further aspect, a method for operating a needle module having the following steps is provided: providing a needle module which comprises a module housing which has a module tip and a front-side module opening formed in the region of the module tip; a puncturing device which has at least one puncturing needle which is arranged on a needle shaft and has a needle tip and is received in the module housing; and a transmission device which is at least partially received in the module housing and has a rolling device; drive-side introduction of a drive movement, which is provided with a repetition frequency, to the transmission device; transmission of the drive movement by means of the transmission device, wherein, in this case, a rolling movement is carried out by means of the rolling device, in which rolling movement a first rolling component of the rolling device, which is mounted rotatably, rolls on a rolling surface on a second rolling component of the rolling device; and coupling of the transmitted drive movement on the output side onto the needle shaft in such a way that the at least one puncturing needle is repeatedly displaced between a retracted position and an extended position by means of a linear movement, wherein the at least one puncturing needle extends through the front-side module opening at least in the extended position and the needle tip is arranged outside the module housing in this case.

The drive movement or drive force, which is provided with the repetition frequency, is transmitted with the aid of the proposed transmission device in an efficient and operationally reliable manner to the needle shaft of the puncturing device and thus finally to the at least one puncturing needle in order to displace the latter between the retracted position and the extended position during operation. The repetitive provision of the drive movement enables a linear repetitive movement of the at least one puncturing needle between the retracted position and the extended position. For this purpose, the transmission device comprises the rolling device in such a way that, by means of the rolling movement between the first and the second rolling component, the drive movement can be transmitted at the repetition frequency, wherein, in this case, the rotatably mounted first rolling component rolls on the rolling surface on the second rolling component. The rolling movement comprises repeated forward and backward rolling of the first rolling component on the rolling surface on the second rolling component in order thus to transmit the drive movement repeatedly or repetitively at the repetition frequency, with the result that the latter can be coupled on an output side from the transmission device to the needle shank and thus to the at least one puncturing needle. The rolling movement which is provided in the transmission device with the aid of the rolling device supports a transmission of the drive movement which is as low-noise as possible.

In the case of the hand-held device, the drive movement or drive force is provided by means of the drive device which is formed, for example, with an electric motor. In this case, the drive device can be received in a handpiece or handpiece module, the housing of which handpiece or handpiece module a user can grip with the fingers when using the hand-held device. The needle module can be connected releasably to the handpiece, as a result of which, for example, a configuration of the needle module as a disposable module is made possible.

The drive movement or drive force, which is provided by the drive device at the repetition frequency, can be coupled into the needle module with the aid of a coupling component, for example a coupling rod or a coupling pin. In this case, the coupling component can extend through a rear-side module opening of the module housing of the needle module. The rear-side module opening can be closed by means of a sealing device, such that an interior of the module housing of the needle module is sealed off with respect to the surroundings on the rear side, for example with the aid of a sealing membrane. The coupling component which couples the drive movement into the needle module can be guided in a sealed manner through a membrane opening of the sealing membrane.

The rolling device can comprise a third rolling component, and, during the transmission of the drive movement, a further rolling movement can be carried out in which the first rolling component rolls on a further rolling surface on the third rolling component. In this embodiment, the first rolling component carries out a plurality of rolling movements on different rolling surfaces and is thus respectively guided on the plurality of rolling surfaces.

For the rolling device, one of the following embodiments can be provided: (i) the second and the third rolling component are arranged on opposite sides in relation to the axis of rotation of the first rolling component; and (ii) the second and the third rolling component are arranged adjacent to one another on the same side in relation to the axis of rotation of the first rolling component. If the first and the third rolling component are arranged on opposite sides of the axis of rotation of the first rolling component, the rolling surfaces provided on the second and on the third rolling component can be arranged opposite one another. If the second and the third rolling component are arranged on the same side and adjacent to one another in relation to the axis of rotation of the first rolling component, the rolling surfaces on the second and on the third rolling component can lie next to one another, whether in the vertical or in the horizontal direction, and can extend in particular parallel to one another in the direction of movement of the rolling movement.

For the rolling device, one of the following embodiments can be provided: (i) the first rolling component is formed with a first wheel component, wherein the rolling device can be operated in such a way that the first wheel component rolls on the rolling surface of the second rolling component during the rolling movement and on the further rolling surface of the third rolling component during the further rolling movement; and (ii) the first rolling component is formed with a first and a second wheel component which are each mounted rotatably on the axis of rotation, wherein the first and the second wheel component have different wheel diameters, and the rolling device can be operated in such a way that the first wheel component rolls on the rolling surface of the second rolling component during the rolling movement and the second wheel component rolls on the further rolling surface of the third rolling component during the further rolling movement.

If the first rolling component is formed with a first wheel component, a tread of the first wheel component comes into contact both with the rolling surface of the second rolling component and with the further rolling surface of the third component during the rolling movements and rolls thereon, respectively, during the transmission of the drive movement.

The first rolling component can be formed with the first and the second wheel component as a double wheel component. The first and the second wheel component can be mounted rotatably on a common axis of rotation. It can be provided that the first and the second wheel component are connected to one another in a rotationally fixed manner, such that a rotation of the first wheel component forces the co-rotation of the second wheel component, and vice versa. The first and the second wheel component roll on the different rolling surfaces during the rolling movements. The rolling surfaces on which the first and the second wheel component roll during the rolling movements can be arranged on opposite sides or on the same side in relation to the axis of rotation of the first rolling component.

The first wheel component can have a smaller wheel diameter than the second wheel component. Thereby, an embodiment is provided for changing a stroke, which is provided with the aid of the drive movement, during the transmission of the drive movement by means of the transmission device, that is to say for increasing or reducing said stroke. For example, the stroke of the drive movement can be at least doubled.

For the rolling device, in one embodiment at least one of the following embodiments can be provided: (i) the rolling surface on the second rolling component is a planar rolling surface; and (ii) the further rolling surface on the third rolling component is a further planar rolling surface. The planar rolling surface can be formed, for example, on a rod or a plate of the respective rolling component.

For the rolling device, in one embodiment at least one of the following embodiments can be provided: (i) during the rolling movement, an axis of rotation of the first rolling component remains in a fixed axial position in the module housing, while the second rolling component with the rolling surface formed thereon is displaced relative to the module housing, wherein the second rolling component is connected to the needle shank for coupling the transmitted drive movement on the output side; and (ii) during the rolling movement, the third rolling component with the further rolling surface formed thereon remains in a fixed component position in the module housing, while the first rolling component with its axis of rotation and the second rolling component with its rolling surface are displaced relative to the module housing, wherein the first or the second rolling component are connected to the needle shank for coupling the transmitted drive movement on the output side.

In the embodiment in which the axis of rotation of the first rolling component remains in the fixed axial position relative to the module housing, the transmitted drive movement is provided on the output side on the second rolling component, in order to then couple said movement to the needle shank, either directly or via one or more intermediate components.

If, on the other hand, the third rolling component remains in the fixed component position relative to the module housing, the drive movement which is transmitted by means of the rolling device is provided on the output side on the first or the second rolling component in a tappable manner. It can be provided here that the drive movement is introduced on the drive side onto the first rolling component, with the result that, for example, the axis of rotation of the first rotary component moves linearly during the rolling movement, in particular back and forth in the longitudinal direction of the module housing.

A transmitted linear movement can be tapped off on the output side, in order to couple it onto the needle shank. In a similar manner, the rolling movement in this case brings about a linear movement of the second rolling component, which can be tapped off on the output side and coupled onto the needle shank.

It may be possible to carry out the linear movements in each case parallel to the longitudinal direction of the module housing.

The rolling device can be operable to displace the third rolling component with the further rolling surface formed thereon relative to the module housing if, during the rolling movement, the axis of rotation of the first rolling component remains in the fixed axial position in the module housing, wherein the second and the third rolling component can be displaced here in opposite directions of movement.

During the displacement of the third rolling component relative to the module housing in the case of a fixed axial position of the axis of rotation of the first rolling component, the third rolling component can carry out a linear movement in the longitudinal direction of the module housing. The movement of the third rolling component relative to the module housing is inverted with the aid of the rolling movements, such that the second rolling component moves in the opposite direction of movement to the movement of the third rolling component.

In this embodiment too, the first rolling component can be formed with the first and the second wheel component, which have different wheel diameters. In this embodiment with the first and the second wheel component, the rolling components moving in opposite directions can be arranged on opposite sides or on the same side in relation to the axis of rotation of the first rolling component, wherein the first wheel component rolls on the rolling surface on the second rolling component and the second wheel component rolls on the further rolling surface of the third rolling component.

The rolling device can be operable to move the second and/or the third rolling component linearly during the rolling movement/the further rolling movement for displacement in the module housing. The respective linear movement can be carried out in the longitudinal direction of the module housing.

For the rolling device, in one embodiment one of the following embodiments can be provided: (i) a drive-side rolling component, onto which the drive movement can be coupled, is formed with the first rolling component, and an output-side rolling component, which can be operated to couple the transmitted drive movement on the output side to the needle shank, is formed with the second or the third rolling component; and (ii) a drive-side rolling component, onto which the drive movement can be coupled, is formed with the second or the third rolling component, and an output-side rolling component, which can be operated to couple the transmitted drive movement on the output side to the needle shank, is formed with the first rolling component.

If the drive-side rolling component is formed with the first rolling component, the drive movement at the repetition frequency is coupled to the first rolling component. On the output side, the transmitted drive movement can be tapped off on the second or the third rolling component and then coupled onto the needle shank.

If the second or the third rolling component forms the drive-side rolling component, the drive movement at the repetition frequency is coupled on the drive side to the second or the third rolling component. On the output side, the transmitting drive movement can be tapped off on the first rolling component, for example in the form of the linear reciprocating movement of the axis of rotation of the first rolling component, in particular in the longitudinal direction of the module housing.

The output-side component, on which the transmitting drive movement can be tapped off, can be formed integrally with the needle shank, for example as an injection-molded component.

For the rolling device, at least one of the following embodiments can be provided: (i) a frictional connection is formed between the first and the second rolling component, and (ii) a frictional connection is formed between the first and the third rolling component. With the aid of the frictional connection, a force fit is formed between the rolling components which participate in the respective rolling movement. To form the frictional connection, anti-slip surfaces, for example rubber surfaces or roughened surfaces, can be provided on the rolling components. The surfaces of the rolling components which come into contact during the rolling movement can be substantially smooth surfaces, in particular tooth-free surfaces.

For the rolling device, in one embodiment at least one of the following embodiments can be provided: (i) a positive connection is formed between the first and the second rolling component, and (ii) a positive connection is formed between the first and the third rolling component.

To form the positive connection, the rolling components which participate can have toothings which are assigned to one another in the region of the surfaces which come into contact during the respective rolling movement. The first rolling component can be, for example, a single or double gearwheel. In one embodiment, the second and/or the third rolling component can be formed with the aid of a toothed rack. Regarding the toothing, a helical toothing or a double helical toothing can be provided. A helical toothing provides in particular an arrangement of the teeth in the gearwheel which is not parallel to the axis of rotation.

The rolling device can be operable to carry out the rolling movement and/or the further rolling movement as pure rolling. The pure rolling ensures, for example, slip-free rolling during the respective rolling movement.

The transmission device can be configured to transmit a linear repetitive drive movement at the repetition frequency. With the aid of the linear repetitive drive movement, a drive-side stroke (drive stroke) can be provided. In relation to the transmission device, a lancing stroke for the extension and retraction of the at least one lancing needle can be provided on the output side, wherein the drive stroke and the lancing stroke can be identical or different with regard to the respective stroke length and/or the respective direction of movement.

By means of the different embodiments of the transmission device, the drive movement (drive stroke) which is provided on the drive side can be transmitted in an inverted and/or scaled manner and coupled onto the puncturing device (lancing stroke).

In one embodiment, the third rolling component can be formed on opposite sides of the first rolling component with the axis of rotation and have a respective section of the further rolling surface, on which the first rolling component rolls during the transmission of the drive movement, on the opposite sides. The second rolling component can also be formed on opposite sides of the first rolling component with the axis of rotation and have a respective section of the rolling surface, on which the first rolling component rolls, on the opposite sides. In such an example, the second and the third rolling component are thus respectively formed on opposite sides of the first rolling component, wherein the first rolling component rolls on the opposite sides on separate sections of the rolling surface and separate sections of the further rolling surface during the transmission of the drive movement.

In one embodiment, the first rolling component can be formed from two wheel components of identical geometry which are independent of one another and which are arranged on the same axis of rotation or different axes of rotation. In one embodiment, both wheel components of the first rolling component can be arranged on axes of rotation which are offset parallel in the direction of movement.

In one embodiment, the needle module can have a return device which is configured or can be operated to provide a return force for the needle shank in order to support or bring about a return movement of the at least one puncturing needle out of the extended position. In this case, provision can be made for the return device to be prestressed against the extension of the at least one puncturing needle into the extended position, with the result that the prestressing force brings about or at least supports the return movement of the at least one puncturing needle out of the extended position. In one embodiment, the return device comprises a tension spring or a compression spring which prestresses the needle shank or the at least one puncturing needle against the movement into the extended position.

In one embodiment, the return device can be formed by means of the sealing membrane which closes off the rear-side module opening in a sealing manner, in such a way that the sealing membrane is elastically stretched or expanded when the at least one puncturing needle is displaced into the extended position, with the result that the elastic membrane force (prestressing force or return force) then retracts the at least one puncturing needle out of the extended position.

In one embodiment, the third rolling component can be formed on opposite sides of the first rolling component with the axis of rotation and have a respective section of the further rolling surface, on which the first rolling component rolls, on the opposite sides. The second rolling component can also be formed on opposite sides of the first rolling component with the axis of rotation and have a respective section of the rolling surface, on which the first rolling component rolls, on the opposite sides. The second and the third rolling component can thus be formed respectively on opposite sides of the first rolling component, wherein the first rolling component rolls on the opposite sides on separate sections of the rolling surface and the further rolling surface. In this case, the first rolling component can be formed in two pieces with an upper first rolling component, in particular an upper first wheel component, and a lower first rolling component, in particular a lower first wheel component, which are mounted rotatably on the common axis of rotation and are arranged above and below a separating sliding surface.

In the different embodiments for transmitting the drive movement, the functions of drive (in particular receiving the drive movement) and output can be interchanged constructively in order to form a transmission ratio which is suitable for the respectively desired application.

The embodiments explained in conjunction with the needle module can be provided correspondingly in conjunction with the hand-held device for locally puncturing a human or an animal skin.

Individual embodiments or a plurality of the embodiments described above for the needle module can also be used correspondingly in the method for operating the needle module.

In one example, a hand-held device for locally puncturing a human skin or an animal skin can be provided, comprising: a needle module and a drive device which is functionally coupled to the needle module in order to transmit a linear repetitive drive movement provided by the drive device to the needle module: the needle module can comprise the following: a module housing which has a module tip and a front-side module opening formed in the region of the module tip; a puncturing device which has at least one puncturing needle which is arranged on a needle shaft and has a needle tip, which puncturing device is received in the module housing in such a way that the at least one puncturing needle can be displaced between a retracted position and an extended position by means of a linear movement, wherein the at least one puncturing needle extends through the front-side module opening at least in the extended position and the needle tip is arranged outside the module housing in this case; and a transmission device. The transmission device can be at least partially received in the module housing and can be operable to transmit the drive movement, which is provided on a drive side with a repetition frequency, to the needle shaft in such a way that the transmitted drive movement can be coupled on an output side to the at least one puncturing needle in order to displace the latter repeatedly between the retracted position and the extended position. The transmission device can have a rolling device which has a first rolling component, which is mounted rotatably, a second rolling component and a third rolling component, wherein the drive movement can be introduced onto the rolling device in such a way that, during the transmission of the drive movement, a rolling movement is carried out in which the first rolling component rolls on a rolling surface on the second rolling component, and a further rolling movement is carried out in which the first rolling component rolls on a further rolling surface on the third rolling component.

### DESCRIPTION OF EMBODIMENTS

Further embodiments are explained below with reference to figures of a drawing. In the figures:

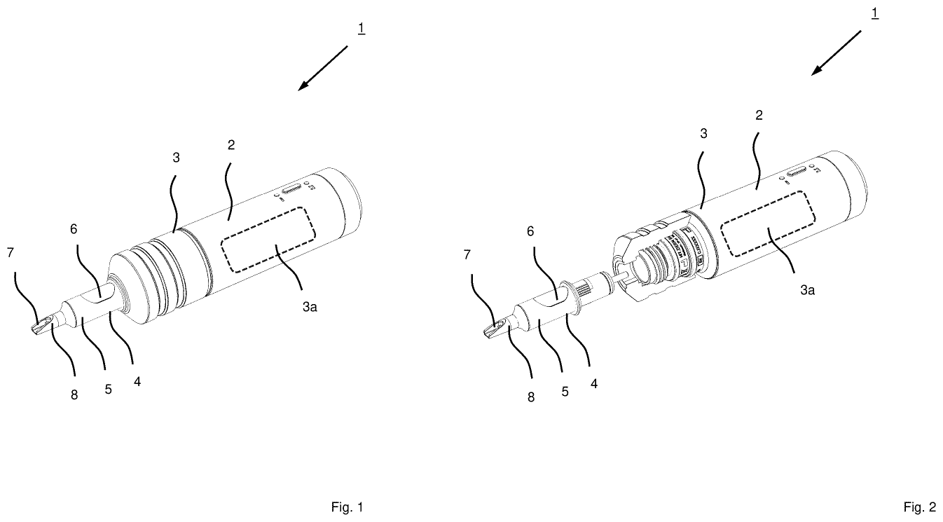

FIG. 1 shows a schematic perspective illustration of a hand-held device for locally puncturing a human or an animal skin;

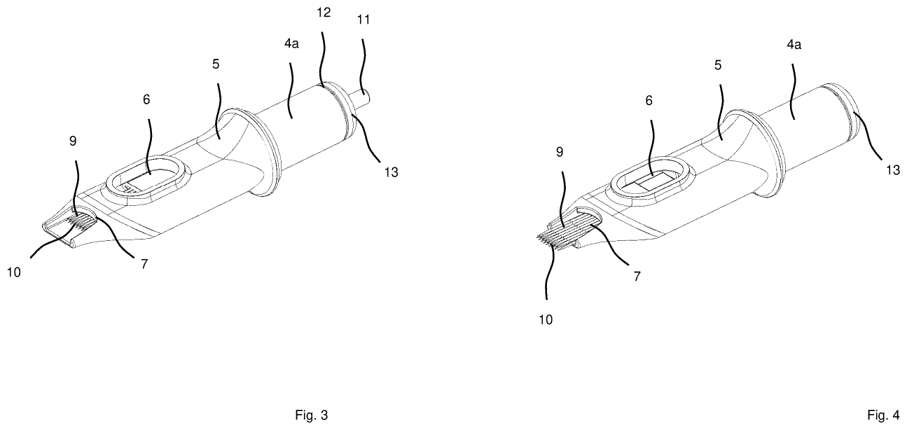

FIG. 2 shows a schematic perspective illustration of the hand-held device from FIG. 1 partially in section, wherein a needle module is shown separately from a handpiece of the hand-held device;

FIG. 3 shows a schematic perspective illustration of a further needle module for a hand-held device for locally puncturing a human or an animal skin with puncturing needles in a retracted position;

FIG. 4 shows a schematic perspective illustration of the further needle module from FIG. 3 with the puncturing needles in an extended position;

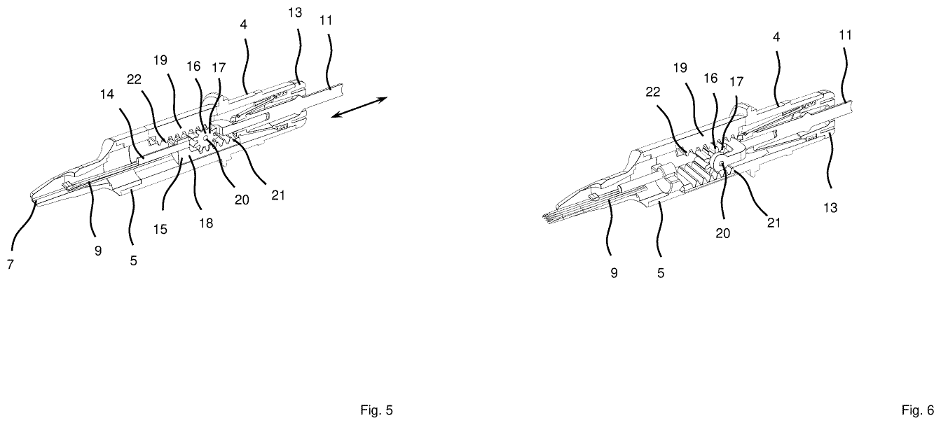

FIG. 5 shows a schematic perspective illustration of a needle module comparable to the embodiment in FIGS. 1 and 2 with puncturing needles in a retracted position in section;

FIG. 6 shows a schematic perspective illustration of the needle module from FIG. 5 with the puncturing needles in the extended position in section;

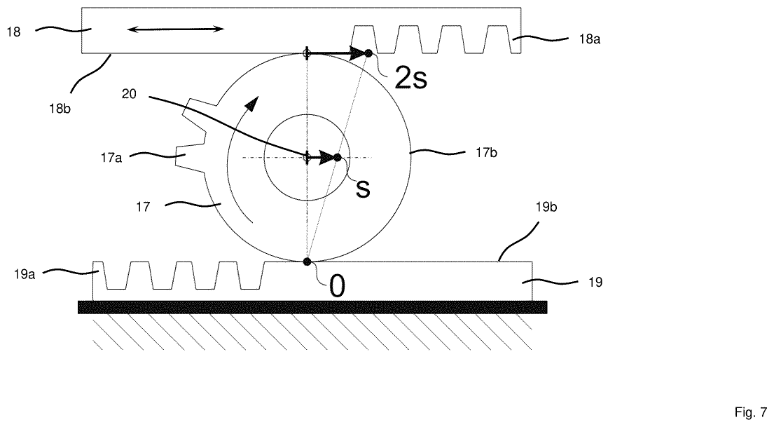

FIG. 7 shows a schematic illustration of an arrangement for a rolling device of a transmission device for transmitting a drive movement which is provided at a repetition frequency;

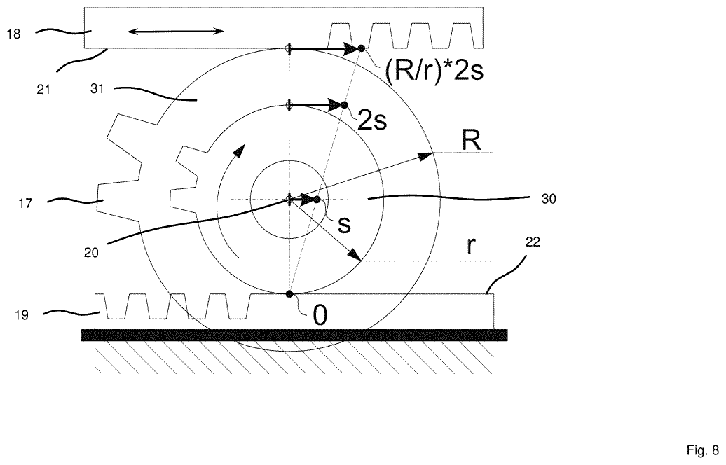

FIG. 8 shows a schematic illustration of an arrangement for a further rolling device of a transmission device for transmitting a drive movement which is provided at a repetition frequency, with a double wheel;

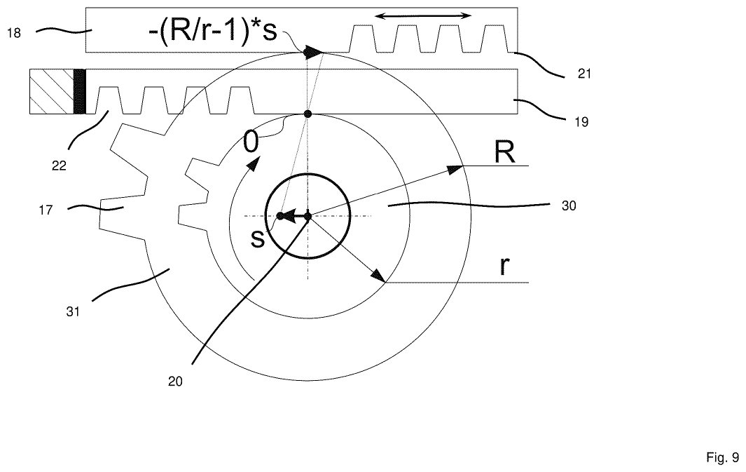

FIG. 9 shows a schematic illustration of an arrangement for an alternative rolling device of a transmission device for transmitting a drive movement which is provided at a repetition frequency, with a double wheel;

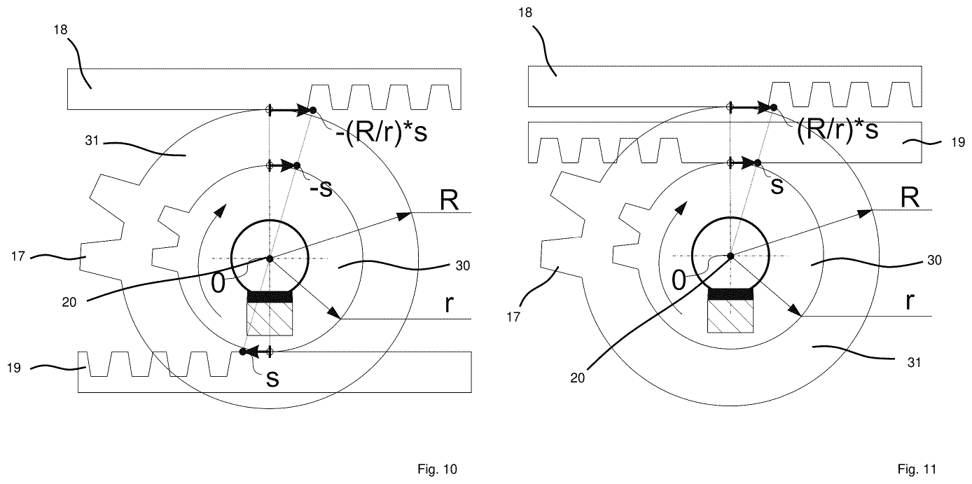

FIG. 10 shows a schematic illustration of an arrangement for another rolling device of a transmission device for transmitting a drive movement which is provided at a repetition frequency, with a double wheel;

FIG. 11 shows a schematic illustration of an arrangement for yet another rolling device of a transmission device for transmitting a drive movement which is provided at a repetition frequency, with a double wheel;

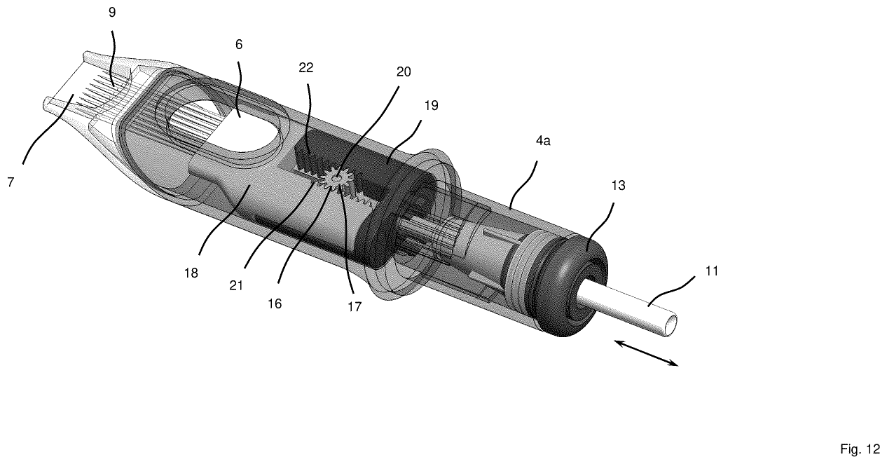

FIG. 12 shows a schematic perspective illustration of the further needle module in the embodiment according to FIG. 3 with puncturing needles in a retracted position;

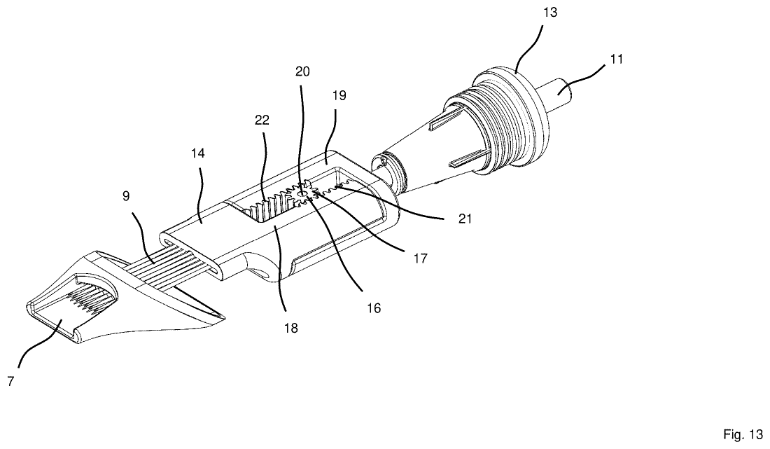

FIG. 13 shows a schematic perspective illustration of the further needle module from FIG. 12, wherein parts of a module housing are omitted;

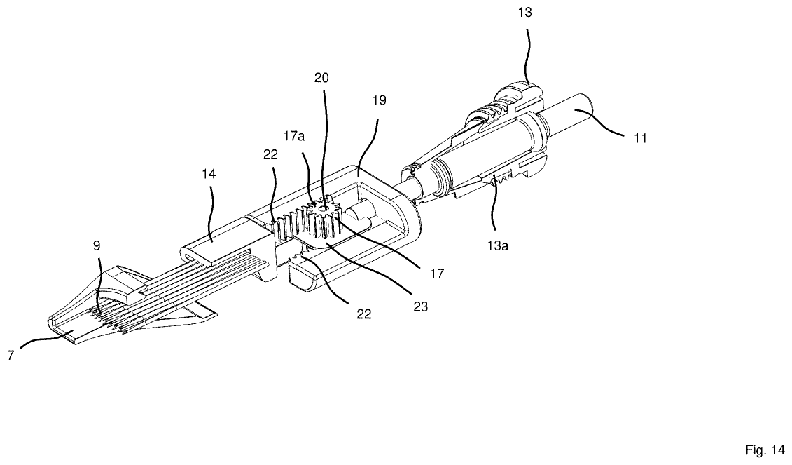

FIG. 14 shows a schematic perspective illustration of the further needle module from FIG. 13 further in detail;

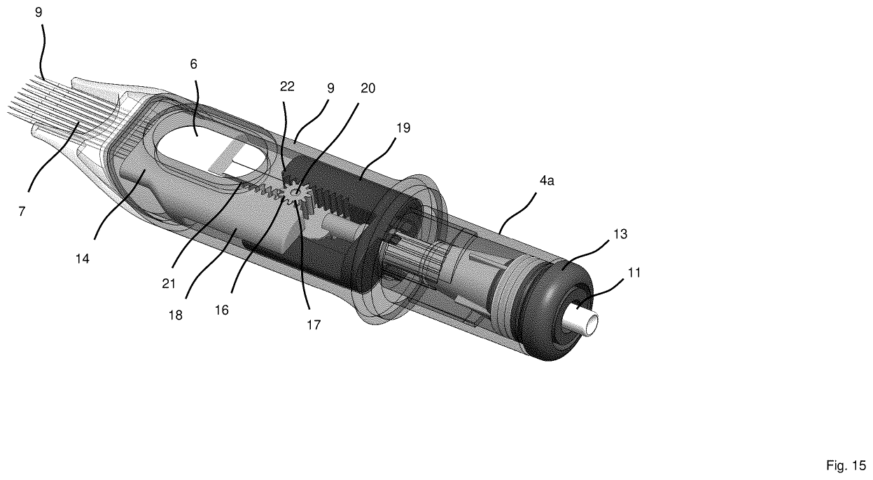

FIG. 15 shows a schematic perspective illustration of the further needle module from FIG. 12 with puncturing needles in the extended position;

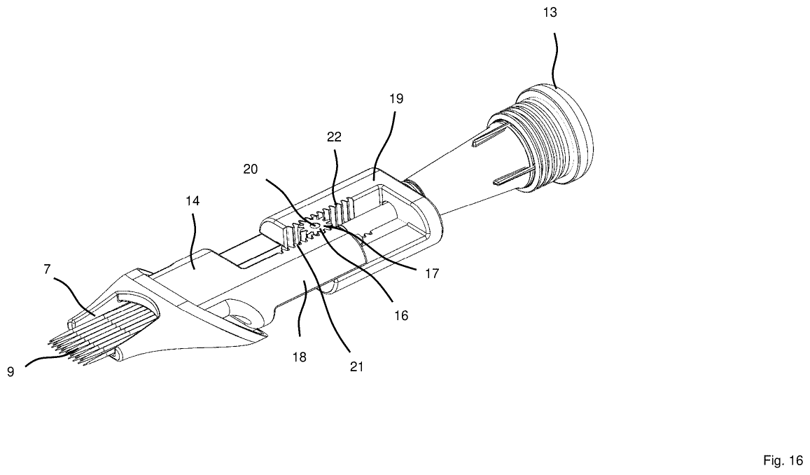

FIG. 16 shows a schematic perspective illustration of the further needle module from FIG. 15, wherein the module housing is partially omitted; and

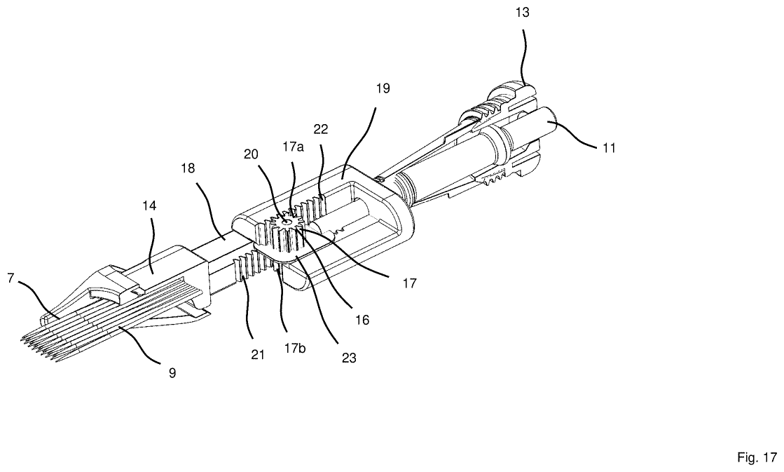

FIG. 17 shows a schematic perspective illustration of the further needle module from FIG. 16 further in detail.

FIGS. 1 and 2 show schematic perspective illustrations of a hand-held device 1 for locally puncturing a human or an animal skin. The hand-held device 1 has a handpiece 2 which can be gripped with the fingers by a user during operation. A drive device 3a, in particular an electric motor, with which a drive movement is provided at a repetition frequency (repetitively) is arranged in a module housing 3 of the handpiece 2. For example, the electric motor can provide a rotational movement which is converted into a linear forward and backward movement with the aid of a conversion mechanism (not illustrated), as is known per se for different embodiments.

A needle module 4 which is formed with a module housing 5 is arranged releasably on the front side of the handpiece 2. The module housing 5, which is formed for example as an injection-molded component, has a filling opening 6 via which a substance can be introduced during operation in such a way that the introduced substance is introduced into the skin during the local puncturing of the latter, for example a dye, a medical or a cosmetic active substance. For this purpose, puncturing needles 9 (cf. FIGS. 3 and 4) are extended and retracted through a front-side module opening 7 at a module tip 8 during operation.

According to FIG. 2, the needle module 4 is arranged releasably on the handpiece 2, such that the needle module 4 can be formed for example as a disposable module which can be removed and disposed of after a puncturing treatment.

FIGS. 3 and 4 show schematic perspective illustrations of a further needle module 4a, wherein the puncturing needles 9 are shown in a retracted position in FIG. 3 and in an extended position in FIG. 4, in which puncturing tips 10 are arranged outside the module housing 5 and protruding with respect to the front-side module opening 7.

For the extension and retraction of the puncturing needles 9, a drive force or drive movement is repeatedly provided by the drive device 3a in the handpiece 2, which drive force or drive movement is transmitted or coupled into the further needle module 4a via a coupling component 11 (cf. FIG. 3). In the embodiment shown, the coupling component 11 extends through a rear-side module opening 12 which is closed and sealed off by means of a sealing membrane 13. The coupling component 11 is guided in a sealed manner through the sealing membrane 13. The sealing membrane 13 is composed of an elastically expandable material, for example a rubber or plastic material. If the coupling component 11 is moved forward in the rear-side module opening 12 in the direction of the front-side module opening 7 on account of the introduction of the drive movement, sections of the sealing membrane 13 are thereby stretched in the direction of the front-side module opening 7. On account of this stretching or expansion, a return force is provided with which the coupling component 11 is retracted after the forward movement, which brings about or at least supports a return movement of the puncturing needles 9 out of the extended position in FIG. 4 into the retracted position according to FIG. 3. In this way, a return device is formed by means of the sealing membrane 13, with which return device a prestressing force is provided against the forward movement of the coupling component 11.

FIGS. 5 and 6 show schematic perspective illustrations of the needle module 4, comparable to the embodiment in FIGS. 1 and 2, with puncturing needles 9 in a retracted position and an extended position, in each case in section. The same reference signs as in the preceding FIGS. 1 to 4 are used for identical features in FIGS. 5 and 6.

It emerges that the puncturing needles 9 are arranged on a needle shank 14 which, during operation, is moved forward and backward in the longitudinal direction of the module housing 5 on account of the drive movement provided at the repetition frequency, with the result that the puncturing needles 9 are repeatedly displaced between the extended position and the retracted position. In the case of the needle module 4 in FIGS. 5 and 6, the module housing 5 has a substantially round outer shape, whereas, in the case of the embodiment in FIGS. 3 and 4, it is formed with a flat shape.

A transmission device 15 is provided for transmitting the linear drive movement, which is coupled via the coupling component 11, onto the needle shank 14. The transmission device 15 comprises a rolling device 16 which, in the example shown, is formed with a first, a second and a third rolling component 17, 18, 19. The first rolling component 17 is mounted rotatably on an axis of rotation 20. The second rolling component 18, which, in the example illustrated, is formed with a toothed rack, is received in the module housing 5 so as to be displaceable in the longitudinal direction of the module housing 5. On the second rolling component 18, a rolling surface 21 is formed opposite the first rolling component 17, which is formed with a gearwheel.

The third rolling component 19, which, in the embodiment shown, is likewise formed with a toothed rack, is mounted fixedly in the module housing 5 and has a further rolling surface 22 which faces the first rolling component 17.

The first rolling component 17 is connected to the coupling component 11 in such a way that the drive movement (drive stroke) can be coupled onto the first rolling component 17, with the result that the latter is displaced with its axis of rotation 20 in the longitudinal direction of the module housing 5 (linear movement), wherein the first rolling component 17 rotates on the axis of rotation 20 in this case, with the result that the second rolling component 18 is displaced in the longitudinal direction of the module housing 5, as shown in FIGS. 5 and 6 for the retracted position and the extended position of the puncturing needles 9. For the transmission of the drive movement, the first rolling component 17 rolls on the rolling surface 21 on the second rolling component 18, and also on the further rolling surface 22 on the third (fixed) rolling component 19, with the result that respective rolling movements are carried out. In the embodiment in FIGS. 5 and 6, a positive connection is formed between the rolling components by means of the gearwheel and the toothed racks.

According to FIG. 6, the second rolling component 18 is moved forward in the module housing 5 on account of the rolling movement as a result of the coupled-in drive movement, as is also the needle shank 14 connected thereto and the puncturing needles 9 arranged on the needle shank 14.

If the coupling component 11 with the first rolling component 17 connected thereto moves back again, the first rolling component 17 rolls back on the further rolling surface 22 of the third rolling component 19, as a result of which, on account of the rolling movement of the first rolling component 17 on the rolling surface 21 of the second rolling component 18, the latter is also moved back in the module housing 5, with the result that finally the puncturing needles 9 are arranged in the retracted position according to FIG. 5.

FIG. 7 shows a schematic illustration of an arrangement for the rolling device 16 with the first, the second and the third rolling component 17, 18, 19. The third rolling component 19 is arranged in a fixed manner in the module housing 5, with the result that an introduction of the drive stroke (repetitive drive movement) onto the first rolling component 17 and a linear movement, brought about thereby, of the axis of rotation 20 by the distance s causes a linear movement of 2 s of the second rolling component 18.

In different embodiments, the first rolling component 17 and the second and the third rolling component 18, 19 can each have a toothing 17a, 18a, 19a (positive connection of the rolling components), which is indicated schematically in FIG. 7 (similarly in FIGS. 8 to 10). Alternatively, the rolling components have tooth-free or smooth surfaces 17b, 18b, 19b (frictional connection of the rolling components) which are assigned to one another for the rolling movement, which is likewise indicated schematically in FIG. 7. In each case, the rolling movements on the rolling surfaces 21, 22 are, for example, pure rolling. The preceding explanations apply correspondingly to further embodiments in the following FIGS. 8 to 10.

FIG. 8 shows a schematic illustration of another arrangement of the rolling device 16, in which the first rolling component 17 is formed with a first wheel component 30 and a second wheel component 31 which have different radii r and R, that is to say different wheel diameters. While the first wheel component 30 rolls on the further rolling surface 22 of the third rolling component 19 during the introduction of the drive movement onto the first rolling component 17, the rolling movement on the rolling surface 21 of the second rolling component 18 takes place by the second wheel component 31. In this way, the drive stroke s, that is to say the linear drive movement which is introduced onto the axis of rotation 20, can be increased to (R/r)×2 s.

FIG. 9 shows a schematic illustration of an alternative arrangement of the rolling device 16, in which the first rolling component 17 is formed with the first wheel component 30 and the second wheel component 31 which have different radii r and R, that is to say different wheel diameters. The second rolling component 18 and the third rolling component 19 are arranged on the same side in relation to the axis of rotation 20 of the first rolling component, above in the shown example. The third rolling component 19 is mounted fixedly. As a result of the introduction of the drive movement onto the first rolling component 17, the first wheel component 30 rolls on the further rolling surface 22 of the third rolling component 19. The rolling movement on the rolling surface 21 of the second rolling component 18 is carried out by the second wheel component 31. In this way, the drive stroke s, that is to say the linear drive movement which is introduced onto the axis of rotation 20, can be changed or translated to −(R/r−1)×s.

Alternatively, the drive movement s can be introduced onto the second rolling component 18 and can be transmitted to the displacement of the axis of rotation 20 of the first rolling component 17 with −r×s/(R−r) (not illustrated).

FIG. 10 shows a further arrangement for an alternative embodiment of the rolling device 16, in which the first rolling component 17 is mounted fixedly in the module housing 5 of the needle module, with the result that, during the rolling movement for the transmission of the drive movement, the second and the third rolling component 18, 19, which are both axially movable, move in the same direction with an opposite sense of direction (inverter arrangement). According to the embodiment in FIG. 10, the second and the third rolling component 18, 19 can be arranged on opposite sides in relation to the axis of rotation 20 of the first rolling component 17.

The examples show that, by means of different embodiments of the transmission device 15, the drive movement (drive stroke) which is provided on the drive side can be transmitted in an inverted and/or scaled manner and can be coupled onto the needle shank 14 (lancing stroke) on the output side.

In the different embodiments for transmitting the drive movement, the functions of drive (receiving the drive movement) and output can be interchanged constructively in order to form a transmission ratio which is suitable for the respectively desired application.

Another arrangement with a fixed position of the axis of rotation 20 of the first rolling component 17 is shown in FIG. 11. Here, the second and the third rolling component 18, 19, which are each arranged displaceably in the module housing 5, are arranged on the same side in relation to the axis of rotation 20.

FIGS. 12 to 17 show schematic perspective illustrations of an arrangement for the further needle module 4a in the embodiment according to FIGS. 3 and 4 with the puncturing needles 9 in the retracted position (cf. FIGS. 12 to 14) and the extended position (cf. FIGS. 15 to 17). The same reference signs as in FIGS. 1 to 6 are used for identical features in FIGS. 12 to 17.

The needle shank 14, on which the puncturing needles 9 are arranged, is connected integrally to the second rolling component 18. During the drive-side coupling of the drive movement, which is transmitted by means of the coupling component, the first rolling component 17 moves with the axis of rotation 20 in the longitudinal direction of the module housing 5 and in this case rolls on the rolling surface 21 and the further rolling surface 22. On account of the fixed position of the third rolling component 19, the second rolling component 18 moves linearly forward and backward. The movement is coupled onto the needle shank 14 on the output side, which needle shank moves together with the second rolling component 18.

If the coupling component 11 is moved forward in the direction of the front-side module opening 7 on account of the coupled-in drive movement, membrane sections 13a of the sealing membrane 13 which surround the coupling component 11 in sections and in turn extend substantially in the longitudinal direction of the module housing are expanded, as a result of which a return force for the return movement of the coupling component 11 and therefore a return movement of the first rolling component 17 is produced.

According to FIG. 14, the third rolling component 19 is formed on opposite sides of the first rolling component 17 with the axis of rotation 20 and has a respective section of the further rolling surface 22, on which the first rolling component 17 rolls, on the opposite sides.

According to FIG. 17, the second rolling component 18 is also formed on opposite sides of the first rolling component 17 with the axis of rotation 20 and has a respective section of the rolling surface 21, on which the first rolling component 17 rolls, on the opposite sides. In the example shown, the second and the third rolling component 18, 19 are thus respectively formed on opposite sides of the first rolling component 17, wherein the first rolling component 17 rolls on the opposite sides on separate sections of the rolling surface 21 and the further rolling surface 22. In this case, the first rolling component 17 is formed in two pieces with an upper first wheel component 17a and a lower first wheel component 17b, which are mounted rotatably on the common axis of rotation 20 and are arranged above and below a separating sliding surface 23.

The features disclosed in the above description, the claims and the drawing can be significant both individually and in any desired combination for the realization of the various embodiments.

1. Needle module for a hand-held device for locally puncturing a human or an animal skin, comprising:
a module housing which has a module tip and a front-side module opening formed in the region of the module tip;
a puncturing device which has at least one puncturing needle which is arranged on a needle shaft and has a needle tip, which puncturing device is received in the module housing in such a way that the at least one puncturing needle can be displaced between a retracted position and an extended position by means of a linear movement, wherein the at least one puncturing needle extends through the front-side module opening at least in the extended position and the needle tip is arranged outside the module housing in this case; and
a transmission device which
is at least partially received in the module housing and can be operated to transmit a drive movement, which is provided on a drive side with a repetition frequency, to the needle shaft in such a way that the transmitted drive movement can be coupled on an output side to the at least one puncturing needle in order to displace the latter repeatedly between the retracted position and the extended position; and
has a rolling device which has a first rolling component, which is mounted rotatably, and a second rolling component and onto which rolling device the drive movement can be introduced in such a way that, during the transmission of the drive movement, a rolling movement is carried out in which the first rolling component rolls on a rolling surface on the second rolling component.

2. Needle module according to claim 1, characterized in that the rolling device has a third rolling component, and in that, during the transmission of the drive movement, a further rolling movement is carried out in which the first rolling component rolls on a further rolling surface on the third rolling component.

3. Needle module according to claim 2, characterized in that one of the following embodiments is provided for the rolling device:
the second and the third rolling component are arranged on opposite sides in relation to the axis of rotation of the first rolling component; and
the second and the third rolling component are arranged adjacent to one another on the same side in relation to the axis of rotation of the first rolling component.

4. Needle module according to claim 2, characterized in that one of the following embodiments is provided for the rolling device:
the first rolling component is formed with a first wheel component, wherein the rolling device can be operated in such a way that the first wheel component rolls on the rolling surface of the second rolling component during the rolling movement and on the further rolling surface of the third rolling component during the further rolling movement; and
the first rolling component is formed with a first and a second wheel component which are each mounted rotatably on the axis of rotation, wherein the first and the second wheel component have different wheel diameters, and the rolling device can be operated in such a way that the first wheel component rolls on the rolling surface of the second rolling component during the rolling movement and the second wheel component rolls on the further rolling surface of the third rolling component during the further rolling movement.

5. Needle module according to claim 4, characterized in that the first wheel component has a smaller wheel diameter than the second wheel component.

6. Needle module according to claim 1, characterized in that at least one of the following embodiments is provided for the rolling device:
the rolling surface on the second rolling component is a planar rolling surface; and
the further rolling surface on the third rolling component is a further planar rolling surface.

7. Needle module according to claim 1, characterized in that one of the following embodiments is provided for the rolling device:
during the rolling movement, an axis of rotation of the first rolling component remains in a fixed axial position in the module housing, while the second rolling component with the rolling surface formed thereon is displaced relative to the module housing, wherein the second rolling component is connected to the needle shank for coupling the transmitted drive movement on the output side; and
during the rolling movement, the third rolling component with the further rolling surface formed thereon remains in a fixed component position in the module housing, while the first rolling component with its axis of rotation and the second rolling component with its rolling surface are displaced relative to the module housing, wherein the first and/or the second rolling component are connected to the needle shank for coupling the transmitted drive movement on the output side.

8. Needle module according to claims 2 and 7, characterized in that the rolling device can be operated to displace the third rolling component with the further rolling surface formed thereon relative to the module housing if, during the rolling movement, the axis of rotation of the first rolling component remains in the fixed axial position in the module housing, wherein the second and the third rolling component can be displaced here in opposite directions of movement.

9. Needle module according to claim 1, characterized in that the rolling device can be operated to move the second and/or the third rolling component linearly during the rolling movement/the further rolling movement for displacement in the module housing.

10. Needle module according to claim 1, characterized in that one of the following embodiments is provided for the rolling device:
a drive-side rolling component, onto which the drive movement can be coupled, is formed with the first rolling component, and an output-side rolling component, which can be operated to couple the transmitted drive movement on the output side to the needle shank, is formed with the second or the third rolling component; and
a drive-side rolling component, onto which the drive movement can be coupled, is formed with the second or the third rolling component, and an output-side rolling component, which can be operated to couple the transmitted drive movement on the output side to the needle shank, is formed with the first rolling component.

11. Needle module according to claim 1, characterized in that at least one of the following embodiments is provided for the rolling device:
a frictional connection is formed between the first and the second rolling component, and
a frictional connection is formed between the first and the third rolling component.

12. Needle module according to claim 1, characterized in that at least one of the following embodiments is provided for the rolling device:
a positive connection is formed between the first and the second rolling component, and
a positive connection is formed between the first and the third rolling component.

13. Needle module according to claim 1, characterized in that the rolling device can be operated to carry out the rolling movement and/or the further rolling movement as pure

14. Needle module according to claim 1, characterized in that the transmission device is configured to transmit a linear repetitive drive movement at the repetition frequency.

15. Hand-held device for locally puncturing a human or an animal skin, comprising a needle module according to claim 1 and a drive device which is functionally coupled to the needle module in order to transmit a drive movement provided by the drive device to the needle module.

16. Method for operating a needle module, comprising
providing a needle module, comprising:
a module housing which has a module tip and a front-side module opening formed in the region of the module tip;
a puncturing device which has at least one puncturing needle which is arranged on a needle shaft and has a needle tip, which puncturing device is received in the module housing; and
a transmission device which is at least partially received in the module housing and has a rolling device;
drive-side introduction of a drive movement, which is provided at a repetition frequency, to the transmission device;
transmission of the drive movement by means of the transmission device, wherein, in this case, a rolling movement is carried out by means of the rolling device, in which rolling movement a first rolling component of the rolling device, which is mounted rotatably, rolls on a rolling surface on a second rolling component of the rolling device; and
coupling of the transmitted drive movement on the output side onto the needle shaft in such a way that the at least one puncturing needle is repeatedly displaced between a retracted position and an extended position by means of a linear movement, wherein the at least one puncturing needle extends through the front-side module opening at least in the extended position and the needle tip is arranged outside the module housing in this case.
